(19) United States
(12) Patent Application Publication
(10) Pub. No.: US 2005/0019378 A1
(43) Pub. Date: Jan. 27, 2005

(54) Transdermal drug delivery system of strychnine, brucine, securinine and their salts

(76) Inventor: Xitian Zhang, Jilin (CN)

Correspondence Address: JACOBSON HOLMAN PLLC, 400 SEVENTH STREET N.W., SUITE 600, WASHINGTON, DC, 20004, US

(21) Appl. No.: 10/496,309

(22) PCT Filed: Oct. 18, 2002
(86) PCT No.: PCT/CN02/00731

(30) Foreign Application Priority Data
Dec. 5, 2001 (CN) 01139894.9

(51) Int. Cl. A61K 31/46; A61K 9/70

(52) U.S. Cl. 424/443; 424/449; 514/304

(57) ABSTRACT

Strychnine, brucine (A) and securinine (B) are a group of natural drugs for central excitation. Because their oral dose or injection dose is close to the half lethal dose, the drugs, with a great toxic side effect, may be lethal if improperly used. The invention is characterized by using the transdermal drug delivery system as a substitute for the oral or injection dosage form to directly act on the local nervous system through the skin, avoiding the toxic side effect caused by drugs directly entering the blood circulating system. The dose through cutaneous absorption is far lower than the oral or injection dose, so it has a much higher safety and a longer acting effect. The transdermal drug delivery system is useful for the treatment of central or peripheral neural paralysis and other nervous system related diseases.

### FIELD OF THE INVENTION

The present invention relates to pharmacology of strychnine, brucine and securinine.

### BACKGROUND OF THE INVENTION

There is a lack of specific drugs for the treatment of central or peripheral neural paralysis and neural dysfunction. From a traditional Chinese viewpoint, “every sort of drug has certain toxicity”. In traditional Chinese medicine, poisonous plant drugs or mineral drugs are often used to treat many difficult and complicated cases of disease, receiving a noticeable curative effect. For example, Nuxvomica and securinega suffruticosa are capable of treating the above diseases.

Strychnine, brucine and securinine extracted from those plants proved potency to treat for the above-mentioned diseases. At present, strychnine and securinine are used as drug only in China.

Whether strychnine and securinine are taken orally or injected, the drug delivery dose is very close to the toxic dose, so it is very unsafe to use them like this. But a change in the route of medication may make it possible to improve the safety and reduce side effects, which will lead to wider use of those drugs.

### SUMMARY OF THE INVENTION

1. Pharmacology of Strychnine, Brucine and Securinine

The natural products involved in the invention are strychnine, brucine and securinine, whose chemical structures are: 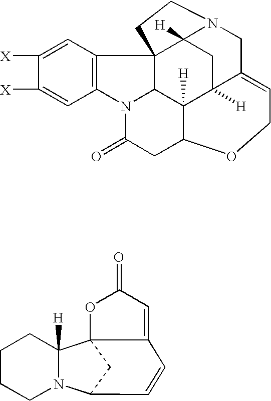
X═H—, strychnine securinine
X═CH₃O—, brucine

Strychnine, brucine and securinine can all excite the spinal reflex function, respiratory center, vagus nerve center, coughing center and vasomotor center. They also can strengthen the excitation process of the cerebral cortex, making the patients in an inhibitory state come to their senses, and improve the functions of the sense organs of taste, touch, hearing and sight.

2. Toxicity of Strychnine, Brucine and Securinine

Excessive strychnine, brucine and securinine can all make the spinal reflex excitation significantly sthenic, causing tetanic spasm and even death from asphyxia due to tetanic contraction of the respiratory muscle.

3 Feasibility of Transdermal Drug

Transdermal drug delivery system can help avoid toxic side effects of the above drugs, but the problem is whether it can use it to achieve the same curative effect. There is a direct correlation between the curative effect of transdermal drug delivery. system and the type of disease. The neurological disease that differs from the viral disease can be treated by stimulating the local nerve, which has an action on the focus through nerve conduction, similar to acupuncture and moxibustion used as the treatment of neurological disease in traditional Chinese medicine. Strychnine, brucine and securinine are drugs for the treatment of neurological disease and they also can achieve the drug delivery effect by acting on local nerves of the human body just like “moxa treatment” in Chinese acupuncture and moxibustion.

The key of this invention is that strychnine, brucine and securinine in vaseline is easy to infiltrate into skin. Vaseline can keep strychnine, bucine and securinine better solubility in vaseline, and it can keep both skin moisture and skin pores bigger. The drug effect of strychnine, brucine/ and securinine in vaseline will be proved.

4. Potency and Safety of Transdermal Drug

1% strychnine ointment is applied to the skin above the facial nerve suffering from central or peripheral facial palsy at the related acupuncture points once a day. Those who suffer from Bell's palsy of 1 to 90 day duration need 1-20 day course of treatment. Those who suffer from facial palsy caused by apoplexy need about 20-40 day duration for treatment. The total effective rate, curative ratio and effective rate are all higher than with acupuncture and moxibustion or Western medicine. Even one who has a course of disease of more than 7 years has also achieved a very good curative effect. The external application experiment involved volunteers showed no skin allergic reaction or other side effects. So transdermal drug is both safe and potency drug.

5 Preparation of Transdermal Drug

Strychnine, brucine and securinine infiltrate through the skin into the body more easily than their salts. They all have certain solubility in vaseline. Vaseline that has a certain action of promoting retention of skin moisture can help increase the rate at which. they infiltrate through the skin. It is easy to make them into ointments or patches.

Some paralysis diseases are related to viral infection. The addition of antibiotics, antifungals and other drugs can help treat for the above diseases at the acute stage, so it is necessary to prepare the transdermal drug with of antibiotics or antifungals.

The infiltration rate of strychnine, brucine and securinine in vaseline is enough, so it is not necessary to use any promoter. Vaseline is a good promoter actually.

The transdermal drug refers to the ointment, plaster and patch. The transdermal drug is easy to be prepared.

6. Method of Application of Transdermal Drug

In order to achieve a better effect of transdermal drug, it is worth recommending the acupuncture point selected in the Chinese techniques of acupuncture and moxibustion. It requires selecting different parts or acupuncture points of the body based on different diseases. For example, to treat for facial palsy, the transdermal drug should be applied to Fengchi, Xiaguan, Taiyang, Jiache and other related acupuncture points. To treat sexual disorder and peripheral non-cream disease, the transdermal drug should be applied to Mingmen, Shenyu, Guanyuan, Baihui and other related acupuncture points.

7. Diseases that Can be Treated with Transdermal Drug of Strychnine, Brucine and Securinine in Vaseline.

The transdermal drug can be used to treat for central or peripheral neural paralysis and neural dysfunction, such as: facial paralysis, hemiplegia, general paralysis, respiratory muscle paralysis, spinal incomplete paralysis, incomplete sphincter, relaxation, prolapse of the penis, Green-Barley syndrome, anal prolapse, peripheral non-cream disease, sexual disorder, sequela of poliomyelitis, myasthenia gravis and amblyopia, postnatal balk anemia.

### DESCRIPTION OF THE PREFERRED EMBODIMENTS

1. Preparation of Ointments

Experiment 1

2-30 mg strychnine and 3 g vaseline are stirred in 50° C. about 10 minutes, which is then put in a package.

2. Preparation of Plaster

Experiment 2

Some of Vaseline containing strychnine, brucine or securinine is applied to the center of a piece of cloth with mucilage glue around the sides.

Industrial Applicability

The preparation for external application is more potency, safe and convenient than the that for oral or injection administration, susceptible of industrial production.

1. The transdermal drug of strychnine, brucine, securinine or their mixtures with or without antibiotics or antifungals in vaseline for the treatment of central or peripheral neural paralysis or neural dysfunction.

2. According to claim 1, the transdermal drug contains strychnine, brucine, securinine or their mixture respectively.

3. According to claim 1, the transdermal drug contains vaseline.

4. According to claim 1, the transdermal drug includes ointment, plaster and patch.

5. According to claim 1, the transdermal drug can contain antibiotics or antifungals for treatment of some diseases.

6. According to claim 1, the transdermal drug can treat for central or peripheral neural paralysis and neural dysfunction, such as: facial paralysis, hemiplegia, general paralysis, respiratory muscle paralysis, spinal incomplete paralysis, incomplete sphincter relaxation, prolapse of the penis, Green-Barley syndrome, anal prolapse, peripheral non-cream disease, sexual disorder, sequela of poliomyelitis, myasthenia gravis and amblyopia, postnatal balk anemia.
